(19) United States
(12) Patent Application Publication
(10) Pub. No.: US 2009/0263505 A1
(43) Pub. Date: Oct. 22, 2009

(54) DIETARY THERAPEUTIC COMPOSITIONS FOR AMELIORATING DISEASE SYMPTOMS AND CONDITIONS ASSOCIATED WITH DIABETES

(76) Inventor: George Roderick Hammons, Mason, OH (US)

Correspondence Address: MICHAEL RIES, 318 PARKER PLACE, OSWEGO, IL, 60543, US

(21) Appl. No.: 12/426,260

(22) Filed: Apr. 19, 2009

Related U.S. Application Data
(60) Provisional application No. 61/046,795, filed on Apr. 21, 2008.

(51) Int. Cl. A61K 33/24; A61K 36/54

(52) U.S. Cl. 424/655; 424/739

(57) ABSTRACT

A daily dietary supplement for improving glucose metabolism, said dietary supplement comprising: an effective amount of a source of Alpha Lipoic Acid; an effective amount of a source of Salacia Oblonga; an effective amount of a source of Gymnema Sylvestre; an effective amount of a source of Chromium; an effective amount of a source of Cinnamon, an effective amount of a source of Banaba; and an effective amount of a source of Fenugreek.

This application claims priority to U.S. Provisional application Ser. No. 61046795, filed 21 Apr. 2008 which is incorporated herein by reference in its entirety.

### BACKGROUND OF THE INVENTION

1. Field of the invention

This invention relates to the use of therapeutic compositions to ameliorate the disease symptoms and conditions associated with diabetes.

2. Description of the Related Art

The present invention is related to a unique supplement for the treatment and prevention of type II diabetes in those individuals with pre-diabetes, or Impaired Glucose Tolerance (IGT). Specifically, the present invention is directed towards a dietary supplement for diabetic control containing a plurality of compounds.

Diabetes has become a leading health care issue in the United States and other industrialized countries, accounting for a relatively large portion of the entire national health care product. The incidence of diagnosed diabetes has increased many fold in America over the past 35 years, with over 24 million diagnosed diabetic patients, another estimated many million undiagnosed diabetic individuals, and still an additional multi-million Americans with pre-diabetes, or Impaired Glucose Tolerance (IGT). As the American populace continues its strong trend towards aging, obesity and greater minority representation, the increasing rate of diagnosed diabetes is certain to continue.

The tremendous economic and physical toll diabetes extracts from society is, in a large part, secondary to both the short and long-term complications of the disease. While there have been great strides made in reducing the short term complications of diabetes, e.g. ketoacidosis, dehydration, and non-ketotic hyperosmolar coma, little, if any, headway has been made in preventing or even minimizing the devastating chronic complications of the disease, e.g. premature atherosclerosis, retinopathy, nephropathy, and neuropathy. Indeed, diabetes has become the leading cause of new cases of blindness in adults in the United States, and now accounts for over a third of all new cases of end-stage renal disease in this country. It is estimated that a diabetic patient's life is shortened by 10 to 15 years, and those years of life are distinguished by a health care cost that is more than three times that of a non-diabetic patient.

Diabetes is a major cardiovascular risk factor, especially among women. This increased risk factor in women is a fact lost by many in both the medical and lay communities. Indeed, a man's risk of dying by heart disease doubles when he develops diabetes, but a woman's risk increases three to five-fold the day she is found to have diabetes. The failure to reduce this increased risk for heart disease over the last seven decades of diabetes management suggests that our current procedures are inadequate.

Type II diabetes, which accounts for over 70% of diabetes, is far more than just a state of abnormal glucose metabolism, but appears to suggest that it is rather a combination of cardiovascular metabolic risk factors, i.e. insulin resistance, hyperinsulinemia, obesity, and hypertriglyceridemia. Much of the excessive cardiovascular mortality associated with diabetes is a result of this array of cardiovascular risk factors, which precedes the onset of diabetes by as much as a decade.

### SUMMARY OF THE INVENTION

In an embodiment there is disclosed therapeutic compositions to ameliorate the disease symptoms and conditions associated with diabetes wherein a daily dietary supplement for improving glucose metabolism comprises:

an effective amount of a source of Alpha Lipoic Acid;

an effective amount of a source of Salacia Oblonga;

an effective amount of a source of Gymnema Sylvestre;

an effective amount of a source of Chromium;

an effective amount of a source of Cinnamon;

an effective amount of a source of Banaba;

an effective amount of a source of Fenugreek.

The foregoing has provided, rather broadly, the preferred ingredients of the present invention so that those skilled in the art may better understand the detailed description of the invention that follows. Additional ingredients of the invention will be identified hereinafter that form the subject of the claim of the invention. Those skilled in the art should appreciate that they can readily use the conception and specific embodiment as a base for modifying the ingredients for carrying out the same purposes of the present invention and that such other modifications do not depart from the spirit and scope of the invention in its broadest form.

### DETAILED DESCRIPTION OF THE INVENTION

In more preferred embodiments, the compositions of this invention employ different components having the same or similar biochemical or therapeutic functionality. These functionally similar components may differ in source, differ in chemical structure and/or different effective half-life on administration. Such combinations of different components with similar activities provide synergistic non-additive benefits and improvements. Components of the compositions of this invention may themselves be multi-component mixtures with each subcomponent having differing functionality. Different composition components may have more than one biological function in the mixture and different components may have distinct, yet overlapping, biological functions. The use of functionally similar components which are structurally distinct or derived from different sources allows the inclusion of sufficiently high levels of total material to achieve a desired level of activity while avoiding the potential toxic effect that may result from use of high levels of any single component.

The present invention is an improved complete formula for managing and maintaining a pre-diabetic condition in a person. The improved formula is comprised of Alpha Lipoic Acid, Salcia Oblonga, Gymnema Sylvestre, Chromium, Cinnamon, Banaba and Fenugreek. The invention reduces the onset of diabetes which, if allowed to occur, could result in severe hyperglycemia which, if uncorrected can result in dehydration, coma and ultimately death.

The formula contains Alpha Lipoic Acid at levels between about 175 mg up to about 350 mg, Salcia Oblonga at levels between about 375 mg up to about 550 mg, Gymnema Sylvestre at levels between about 275 mg up to about 450 mg, Chromium at levels between about 275 mg up to about 450 mg, Cinnamon at levels between about 850 mg up to about 1200 mg, Banaba at levels between about 10 mg up to about 60 mg, and Fenugreek at levels between about 300 mg up to about 600 mg. Suitable sources of Chromium can include Chromium yeast,

Chromium acetate, Chromium Polynicotinate and Chromium Picolinate. The preferred source is Chromium Polynicotinate at levels between about 100 mcg up to about 250 mcg.

Table 1 contains the approximate preferred levels of the ingredients by amount of the specific formulas for use in the prevention of diabetes and diabetic complications:

TABLE 1
Ingredients Approximate Preferred Amount to be taken daily.

| | |
|---|---|
| 300 | mg of Alpha Lipoic Acid, |
| 500 | mg of Salacia Oblonga, |
| 400 | mg of Gymnema Sylvestre, |
| 200 | mcg of Chromium Polynicotinate, |
| 1060 | mg of Cinnamon, |
| 500 | mg of Fenugreek, |
| 40 | mg of Banaba |

Alpha Lipoic Acid is nutritional support for healthy glucose metabolism.

Salacia Oblongais a herb used to support healthy blood glucose levels in adults.

Gymnema Sylvestreis a plant native to the tropical forests of India. It has been recognized for its ability to increase cell permeability.

Chromium Polynicotinate is an essential mineral nutrient that is vital to blood sugar metabolism. It works to facilitate the uptake of glucose into cells.

Cinnamon has been shown to stimulate glucose uptake and glycogen synthesis.

Fenugreek is a food and a spice commonly eaten in many parts of the world.

Banaba is a medicinal plant that grows in India, Southeast Asia and the Philippines. The effect of Banaba Leaf extract induces glucose transport from the blood into body cells.

The components of the formula may be contained together within a vegetable capsule. For the purposes of oral administration, the compositions of the invention may be put up in unit dosage form or in larger dosages. In so far as the compositions are liquid this may be done by filling a capsule, such as a vegetable capsule.

Although an example of the present invention has been described, it would be appreciated by those skilled in the art that changes might be made in this embodiment without departing from the principles and spirit of the invention, the scope of which is defined in the claims and their equivalents.

What is claimed is:

1. A daily dietary supplement for improving glucose metabolism, said dietary supplement comprising:
an effective amount of a source of Alpha Lipoic Acid;
an effective amount of a source of Salacia Oblonga;
an effective amount of a source of Gymnema Sylvestre;
an effective amount of a source of Chromium;
an effective amount of a source of Cinnamon.
an effective amount of a source of Banaba; and
an effective amount of a source of Fenugreek.

2. The daily dietary supplement of claim 1 wherein said effective amount of chromium further comprises:
Chromium Polynicotinate.

3. A daily dietary supplement according to claim 2, wherein said effective amount of a source of Chromium Polynicotinate is in the range of about 100 mcg up to about 250 mcg.

4. A daily dietary supplement according to claim 2, wherein said effective amount of a source of Banaba is in the range of about 10 mg up to about 60 mg.

5. A daily dietary supplement according to claim 2, wherein said effective amount of a source of Fenugreek is in the range of about 300 mg up to about 600 mg.

6. A daily dietary supplement according to claim 2, wherein said effective amount of a source of Alpha Lipoic Acid is in the range of about 175 mg up to about 350 mg.

7. A daily dietary supplement according to claim 2, wherein said effective amount of a source of Salacia Oblonga is in the range of about 375 mg up to about 550 mg.

8. A daily dietary supplement according to claim 2, wherein said effective amount of a source of Gymnema Sylvestre is in the range of about 275 mg up to about 450 mg.

9. A daily dietary supplement according to claim 2, wherein said effective amount of a source of Cinnamon is in the range of about 700 mg up to about 1200 mg.

10. A daily dietary supplement according to claim 2, wherein said effective amount of said source of Alpha Lipoic Acid is about 300 mg.

11. A daily dietary supplement according to claim 2, wherein said effective amount of said source of Salacia Oblonga is about 500 mg.

12. A daily dietary supplement according to claim 2, wherein said effective amount of said source of Gymnema Sylvestre is about 400 mg.

13. A daily dietary supplement according to claim 2, wherein said effective amount of said source of Chromium Polynicotinate is about 200 mcg.

14. A daily dietary supplement according to claim 2, wherein said effective amount of said source of Cinnamon is about 1060 mg.

15. A daily dietary supplement according to claim 2, wherein said effective amount of said source of Fenugreek is about 500 mg.

16. A daily dietary supplement according to claim 2, wherein said effective amount of said source of Banaba is about 40 mg.

17. A daily dietary supplement for improving glucose metabolism, said dietary supplement comprising:
an amount of about 300 mg of Alpha Lipoic Acid;
an amount of about 500 mg of Salacia Oblonga;
an effective amount of about 400 mg of Gymnema Sylvestre;
an effective amount of about 200 mcg of Chromium Polynicotinate;
an effective amount of about 1060 mg of Cinnamon.
an effective amount of about 40 mg of Banaba; and
an effective amount of about 500 mg of Fenugreek.
